(19) United States
(12) Patent Application Publication
(10) Pub. No.: US 2013/0327329 A1
(43) Pub. Date: Dec. 12, 2013

(54) METHOD AND ARRANGEMENT FOR GENERATING A GAS STREAM COMPRISING NITRIC OXIDE

(71) Applicants: Claudia Hannelore Igney, Erlangen (DE); Achim Koerber, Eindhoven (NL); Rainer Hilbig, Aachen (DE)

(72) Inventors: Claudia Hannelore Igney, Erlangen (DE); Achim Koerber, Eindhoven (NL); Rainer Hilbig, Aachen (DE)

(73) Assignee: KONINKLIJKE PHILIPS N.V., EINDHOVEN (NL)

(21) Appl. No.: 13/984,356

(22) PCT Filed: Feb. 16, 2012
(86) PCT No.: PCT/IB2012/050716

§ 371 Date: Aug. 27, 2013

(30) Foreign Application Priority Data
Feb. 21, 2011 (EP) 11155126.3

(51) Int. Cl. A61M 16/12

(52) U.S. Cl. CPC A61M 16/12
USPC 128/203.14; 128/203.12

(57) ABSTRACT

The invention relates to the field of nitric oxide generation. The present invention particularly relates to a method for generating a stream of gas, the gas comprising oxygen and nitric oxide. According to the invention, an intermittent stream of oxygen comprising gas having at least one gap is provided, in which gap a defined pulse of nitric oxide comprising gas is provided. The pulse of nitric oxide comprising gas is embedded in a first pulse of inert gas being provided before the pulse of nitric oxide comprising gas and a second pulse of inert gas being provided after the pulse of nitric oxide comprising gas, the first pulse of inert gas and the second pulse of inert gas being provided in the gap of the intermittent stream of oxygen comprising gas. The method according to the invention reduces the danger of toxic compounds to be formed in the generated gas stream and furthermore provides a method being usable for therapeutic applications independent from the breathing cycle of a patient.

### FIELD OF THE INVENTION

The invention relates to the field of nitric oxide application. More particularly, the invention relates to the field of therapeutic nitric oxide application by inhalation.

### BACKGROUND OF THE INVENTION

It is widely known to use nitric oxide (NO) in a variety of applications. Next to technical applications such as an intermediate in the Ostwald process for the synthesis of nitric acid from ammonia, especially several therapeutic applications using nitric oxide are known.

One of the most famous therapeutic applications of nitric oxide is the administration for neonates suffering from Persistent Pulmonary Hypertension (PPHN). However, many comparable or other therapeutic applications are known and discussed for the use of nitric oxide. As an example, nitric oxide is used by the endothelium of blood vessels to signal the surrounding smooth muscle to relax, thus resulting in widening the blood vessels and therefore increasing blood flow. This leads to nitric oxide being particularly applicable for the therapy of hypertension. Further exemplary applications for nitric oxide are directed towards improving lung function and treating or preventing bronchoconstriction, reversible pulmonary vasoconstriction, or for treating or preventing arterial restenosis resulting from excessive intimal hyperplasia. These applications mostly involve inhalation of a stream of nitric oxide comprising gas.

The administration of nitric oxide (NO) to patients may however cause difficulties. Due to the fact that nitric oxide tends to react with oxygen (O₂), toxic nitrogen oxides in higher oxidation states may be formed. For example, nitrogen dioxide (NO₂) may be formed by nitric oxide reacting with oxygen. Especially the formation of nitrogen dioxide has to be minimized or avoided due to is high toxicity even in very low concentrations. Consequently, there is the dilemma that nitric oxide has to be administered in a concentration being high enough to achieve the desired effect, but being low enough to avoid or to minimize nitrogen dioxide to be formed.

In order to solve this problem, known from WO 95/10315 A1 is a nitric oxide treatment which particularly concerns the use of nitric oxide in the treatment of certain lung diseases. This nitric oxide treatment is mainly based on delivering nitric oxide to the lungs by inhalation, wherein it is often not completely avoidable that oxygen mixes with the inhaled nitric oxide gas due to the fact that gas masks are used by the patient. Known from this document is thus a method of treatment in which nitric oxide is administered to a patient not continuously, but intermittently and in short pulses of known, pre-determined volume at one or more suitable times during each inhalation. These pulses of nitric oxide are thereby delivered at the end or at the beginning of each breathing cycle of the patient, wherein the breathing cycle may be controlled by a sensor being located in a mask. The pulses are administered either together with, or preferably side by side with a supply of air, oxygen or oxygen-enriched air.

However, even if there is a certain division of nitric oxide and oxygen in the generated gas stream at a time directly after introducing the nitric oxide pulse, there is the risk of nitric oxide to dilute into the oxygen phase by gas diffusion or vice versa resulting in the danger of nitrogen oxides to be formed.

Additionally, due to the fact that the nitric oxide pulse has to be provided either at the beginning or at the end of a breathing cycle, it is essential to control the breathing cycle. This requires a sensor to be used which may lead to a cost intensive method. Additionally, if the sensor fails, there may be the risk of even higher amounts of nitrogen oxides to be formed during inhalation.

### SUMMARY OF THE INVENTION

It is an object of the present invention to provide a method for generating a stream of gas comprising nitric oxide which overcomes at least one of the limitations as set forth above.

It is a further object of the present invention to provide a method for generating a stream of gas comprising nitric oxide which further reduces the formation of nitrogen oxides and which may be used in therapeutic applications independent from the breathing cycle of the patient.

These objects are achieved by a method for generating a stream of gas, the gas comprising oxygen and nitric oxide, wherein an intermittent stream of oxygen comprising gas having at least one gap is provided, in which gap a pulse of nitric oxide comprising gas is provided, wherein the pulse of nitric oxide comprising gas is embedded in a first pulse of inert gas being provided before the pulse of nitric oxide comprising gas and a second pulse of inert gas being provided after the pulse of nitric oxide comprising gas, the first pulse of inert gas and the second pulse of inert gas being provided in the gap of the intermittent stream of oxygen containing gas.

According to the invention, the danger of nitric oxide to be oxidized due to a reaction with oxygen being present in the oxygen comprising gas is addressed by providing an intermittent stream of oxygen comprising gas having at least one gap. This shall mean that the stream of oxygen comprising gas is not provided in a continuous way, but there is at least one gap, or break, respectively, in which no oxygen comprising gas will be provided. It is apparent that, according to the duration of the gas stream, a plurality of gaps may be provided. The gap or gaps may be defined in a temporary point of view as well as by length due to the flowing character of the gas stream. Preferably, there are provided a plurality of gaps in the intermittent flow of oxygen comprising gas.

In each gap, a pulse of nitric oxide comprising gas is provided. The nitric oxide comprising gas may thereby be pure nitric oxide or preferably a mixture of nitric oxide in a carrier gas. This may be preferred as for many applications a limited concentration of nitric oxide is required. The nitric oxide comprising gas may thereby be provided, or introduced into the stream of oxygen comprising gas, or in its gaps, respectively, by a valve or the like. Consequently, the nitric oxide comprising gas is divided from the oxygen comprising gas by time and consequently by place with respect to the whole gas being conveyed through a certain distance. This already allows reducing nitric oxide to be oxidized.

Additionally, according to the invention, the pulse of nitric oxide comprising gas is flanked by, or embedded in, respectively, two pulses of inert gas. This additional measure has the effect that the nitric oxide comprising gas is not in direct contact to the oxygen comprising gas, thereby anyhow allowing a continuous stream of gas. Consequently, oxygen is prevented from coming in contact with nitric oxide, or the latter is at least considerably reduced, resulting in even more minimizing the danger of nitric oxide to be oxidized.

This beneficial effect is thereby achieved not only directly after generating the respective gas stream, but also after a certain distance the generated gas stream was conveyed, i.e. after a certain level of time the gas was flowing. Under these circumstances, it may sometimes not completely be avoided, that the respective gas phases will be intermixed in a certain and limited extend. For example, it may be possible that the respective gas diffuses through the phase border and will thus be diluted in an adjacent gas phase. However, according to the invention, if oxygen, for example, will diffuse through the gas phase border, it will at first be intermixed in the inert gas pulse. Correspondingly, if nitric oxide will diffuse through the phase border, it may at first diffuse into the inert gas pulse, but not into the oxygen comprising gas. Additionally, even if both nitric oxide and oxygen will diffuse into the inert gas pulse, or phase, respectively, the concentration will be far too low to result in a fast reaction of oxygen and nitric oxide because this reaction is strongly dependent from the concentration of both gases. Furthermore, even if these both gases will anyhow react, the concentration of the formed nitrogen oxides, in particular nitrogen dioxide will be far too low to present a considerable security risk. This is even the case for therapeutic applications of the so formed stream of gas.

Consequently, the pulse of inert gas forms a diffusion barrier for oxygen and/or for nitric oxide which minimizes or completely avoids a contact of nitric oxide and oxygen and thus minimizes or completely avoids a considerable generation of nitrogen dioxide.

The pulse of oxygen comprising gas, the pulse of nitric oxide comprising gas as well as the pulse of inert gas may thereby be provided in, or introduced into a conduit at one place. This allows providing sharp borders of the respective pulses, or gas phases, respectively, minimizing the danger of mixing the respective phases right at the beginning and thus optimizing the effect of the inert gas pulses. The stream, i.e. the flowing gas, may thereby be provided simply by pressing the respective gases into a conduit. This may be realized by a suitable pump, or by providing pressurized gas storing devices, for example.

In a preferred embodiment of the present invention the stream of gas is provided in a gas administration device for therapeutic applications. This shall mean that the generated gas stream is conveyed to the gas administration device, or that it is at least partly formed therein. This embodiment is especially preferred as there are many therapeutic applications in which a gas comprising oxygen and nitric oxide is administered. According to this embodiment, one main benefit of the present invention becomes apparent. Due to the fact that the nitric oxide pulse is embedded in two inert gas pulses, administration to a patient may be performed independent from the breathing cycles of the latter. Consequently, the provision of a sensor which detects breathing, or inhalation, respectively, of the patient is not necessary. This allows performing the method according to the invention in a cost saving manner and more facile. In fact, an observation of the breathing cycle may be omitted which makes the method according to the invention to be performed without any nursing staff, for example. Additionally, a component being present has always the danger of a failure. Consequently, due to the fact that no sensor for detecting the breathing cycle is present, the function of the latter has not to be controlled and the risk of a failure and consequently a deficient administration to the patient is prevented. Preferably, the gas stream is generated close to a gas administration device in order to further reduce gas diffusion of the respective phases on their short way to the patient.

In a further preferred embodiment of the present invention the gas stream exhibits a Reynolds number of ≦4000, in particular of ≦2300. The Reynolds number in an air duct is defined as Re=Q*D_{H}/(v*A) where Q=volumetric flow rate (m³_{SATP}/s), D_{H}=hydraulic diameter of the duct (m), ν=kinematic viscosity of air (m²/s) and A=duct cross-sectional area (m²). This allows the gas stream to have a substantially laminar flow because of which turbulences are reduced or completely avoided. Consequently, diffusion of the respective pulses or phases is furthermore reduced and thus the danger of a reaction of nitric oxide and oxygen is further reduced, or completely avoided. It is preferred, that the gas stream exhibits the above defined Reynolds number in a conduit in which the gas stream comprising the different pulses of oxygen containing gas, inert gas and nitric oxide comprising gas are generated, or conveyed in, respectively. Consequently, as far as therapeutic applications are concerned, at least at a point of time at which a patient inhales the generated gas, no or very limited intermixture has taken place and thus no or no considerable amount of nitrogen oxides are formed. However, depending of the inhalation device a patient uses, the generated gas stream may be provided in the trachea in the desired way, i.e. with a nitric oxide comprising gas being completely or at least substantially completely embedded in two inert gas pulses. Consequently, a reaction of nitric oxide may be reduced even in the trachea and thus until the patient absorbs the respective gases, or exhales them.

In a further preferred embodiment of the present invention air or oxygen is used as oxygen comprising gas. This allows to combine, for example, a therapy based on nitric oxide administration with a therapy comprising oxygen administration especially if oxygen if used. However, if air is used as oxygen comprising gas, the generated gas stream may solely be used as breathing atmosphere for a patient. Consequently, the gas stream may be administered to the patient and the patient may breathe in a normal cycle, thereby getting a therapeutic amount of nitric oxide as well as an appropriate amount of air, or oxygen, respectively.

In a further preferred embodiment of the present invention the oxygen comprising gas is air and a first pulse of oxygen is provided before the first inert gas pulse and a second pulse of oxygen is provided after the second inert gas pulse, the first and the second oxygen pulses being provided in the gap of air. This allows a combined administration of both oxygen and nitric oxide to a patient, thereby being based on an administration of air for normal breathing purposes.

In a further preferred embodiment of the present invention nitrogen is used for providing the first and the second inert gas pulse. This allows using a cost-efficient inert gas the handling of which is unproblematic. Additionally, diffusion of nitrogen either in the phase of nitric oxide comprising gas or in the phase of oxygen comprising gas does not lead to any problem.

In a further preferred embodiment of the present invention a mixture of nitrogen and nitric oxide is used as nitric oxide comprising gas. This gas mixture may preferably be used for a variety of applications, in which nitric oxide in lower concentrations is required. Additionally, this mixture may be provided cost-saving and may be stored, at least for a limited period of time, without the danger of nitrogen oxides to be formed. Apart from that, in this embodiment it is especially possible to get further synergistic effects. For example, it is possible in combination with nitrogen being used for the inert gas pulse, to only use one source of nitric oxide, one source of oxygen and one source of nitrogen. The mixture of nitrogen and nitric oxide may in this case be generated by introducing a respective amount of each gas into a conduit during a gap of the oxygen containing gas stream. Correspondingly, the oxygen comprising gas may be formed by using nitrogen and oxygen. Consequently, the method according to this embodiment of the present invention may be carried out with smaller gas cylinders and may furthermore be adjusted, for example with respect to the desired gas concentrations, even during performing the method according to the present invention. This may be preferred for therapeutic applications and especially for homecare devices, because weight as well as room may be saved.

In a further preferred embodiment of the present invention the gas stream exhibits a SATP flow rate in the range of ≧0,01 L_{SATP}/min to ≦10 L_{SATP/}min, in particular of 0,4 L_{SATP}/min, wherein “L_{SATP}” means the amount of gas in 1 L volume at standard ambient temperature (25° C.; 298,15K) and pressure (1 bar). These flow rates are very well suited for a direct administration of the generated gas stream comprising nitric oxide without the requirement of (pre-) storing it. Of course, low flow rates are preferred as this further minimizes the danger of a mixture of the different phases. It becomes apparent to one skilled in the art that the flow rates of the nitric oxide comprising gas, the respective inert gas pulses as well as the pulse of the oxygen comprising gas have to be in the same range in order to get a well defined continuous gas flow. This however is well achievable by defining the conditions of an insertion of the respective pulses. Of course, additionally to the flow rate of the gas stream, the concentration especially of the nitric oxide comprising gas may be varied according to the desired application. For example, nitric oxide may be provided in the nitric oxide comprising gas in a concentration lying in the range of ≧1 ppm to ≦500 ppm, especially preferred in a range of ≧10 ppm to ≦100 ppm. In detail, if the generated nitric oxide comprising gas stream shall be used in therapeutic applications, there are different parameters to be met. In detail, if the gas is used for respiratory applications, e.g. PPHN, quite high air flows in the range of 6 L/min with moderate average concentrations of nitric oxide in the range of 20-40 ppm are required. The average concentration shall thereby mean the concentration of nitric oxide with respect to the whole air stream.

It is furthermore preferred that the pulse width Wₚᵤₗₛₑ of each inert gas pulse lies in the range of ≧1 cm to ≦10 cm, preferably in the range of ≧4 cm to ≦6 cm, further preferred at 5 cm. These pulse widths provide a barrier which is wide enough to inhibit or at least to significantly reduce the oxygen to dissociate into the nitric oxide phase, or vice versa.

It becomes apparent to one skilled in the art that these pulse widths are adjustable according to any desired application. They are easily adjustable by taking into consideration the flow rate Q of the gas stream as well as the cross-sectional area A of the conduit the stream of gas being provided in:

$$\frac{{W}_{pulse}}{{T}_{pulse}} = V = \frac{Q}{A}$$

wherein Tₚᵤₗₛₑ is the temporal length of the pulse and V the mean gas velocity.

The invention further relates to an arrangement for providing an oxygen and nitric oxide comprising gas stream, the arrangement comprising a first gas source for a nitric oxide comprising gas, a second gas source for an oxygen comprising gas, and a third gas source for an inert gas, wherein each of the first gas source, the second gas source, and the third gas source are connected to a conduit, wherein a flow regulation device is provided for each gas source to allow a flow of the gas into the conduit to be adjusted, and wherein the arrangement further comprises a controlling device for controlling the flow regulation device such, that a stream of gas is generated according to a method according to the invention.

The arrangement according to the invention is thus designed to perform a method according to the invention and thus exhibits the advantages like described above with respect to the method.

The conduit may thereby be any conduit which is suitable for guiding a gas flow. For example, the conduit may be a duct, a pipe, a tubing, or the like. Especially for therapeutic applications, it may be preferred that a flexible tube is used, which may for example be formed of plastic material.

The flow regulation device may be any means which is suitable for enabling or for inhibiting a flow of gas, completely or partly. For example, the flow regulation device may be a valve.

The respective gas sources may be any source which may provide the desired gas. For example, the gas sources may be gas storing devices, such as gas cylinders. Alternatively, the gas sources may be devices which may generate the desired gas in-situ, in which case the gas generation devices are preferably connected to a gas reservoir to ensure that the desired amount of gas may be provided at any time.

The controlling device for controlling the regulation device such, that a stream of gas is generated according to the method according to the invention may be designed as is clear to one skilled in the art. As an example, a controlling device may be provided in which respective pulse sequences may be introduced and saved. The controlling device will then act on the respective flow regulation device and will open or close the connection of the respective gas source in order to let this gas egress into the conduit, or inhibit the latter or to adjust the amount of egress. By selectively opening or closing or regulating the amount of gas being egressed, the respective pulses may be generated and thus the respective pulse sequence may be generated in the required concentration.

### BRIEF DESCRIPTION OF THE DRAWINGS

These and other aspects of the invention will be apparent from and elucidated with reference to the embodiments described hereinafter.

In the drawings:

FIG. 1 shows a schematic view of an arrangement configured for performing the method according to the invention;

FIG. 2 shows a defined pulse sequence suitable for performing the method according to the invention;

FIG. 3 shows a simulation of the concentration distribution of the gases according to FIG. 2 directly after generation of the pulse sequence (FIG. 3a) and after a certain level of conveyance (FIG. 3b);

FIG. 4 shows a further defined pulse sequence suitable for performing the method according to the invention; and

FIG. 5 shows a simulation of the concentration distribution of the gases according to FIG. 4 directly after generation of the pulse sequence (FIG. 5a) and after a certain level of conveyance (FIG. 5b).

### DETAILED DESCRIPTION OF EMBODIMENTS

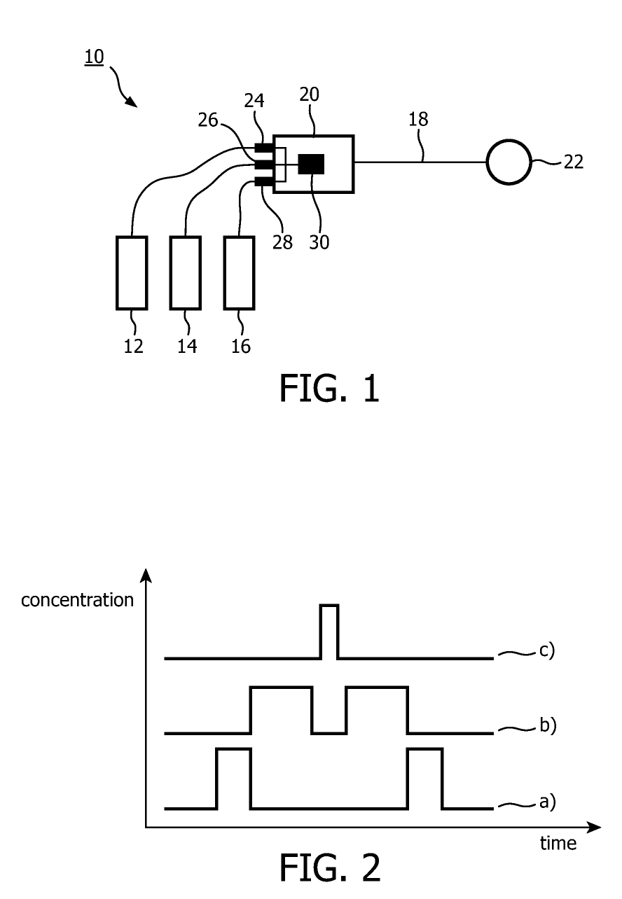

In FIG. 1, an arrangement 10 according to the invention is shown which is adapted for carrying out the method according to the invention. In detail, the arrangement 10 comprises a first gas source 12 for a nitric oxide comprising gas, a second gas source 14 for an oxygen comprising gas and a third gas source 16 for an inert gas. The gas sources 12, 14, 16 are connected to a conduit 18, for example via a gas introduction section 20. This allows an easy insertion of the respective gas streams one after the other in form of pulses, thereby enabling sharp borders. The conduit 18 may be designed as a tubing or the like and it may be connected to a gas administration device 22. The gas administration device 22 may be designed in dependence of the desired application of the generated gas stream. When used for therapeutic applications, for example, the gas administration device 22 may be formed as a mask.

The respective gases as well as the conduit 18 and the gas administration device 22 are preferably formed to allow the generated stream of gas to exhibit a Reynolds number in the range of ≦4000, preferably in the range of ≦2300. This enables the generated gas stream to form a substantially laminar gas flow. For the example of a cylindrical air duct

$$A = \frac{π}{4} \cdot {D}_{H}^{2}$$

and the Reynolds number Re becomes

$$Re = \frac{4}{π \cdot v} \cdot \frac{Q}{{D}_{H}} .$$

This implies that for a given flow rate Q a sufficiently low Reynolds number (to guarantee a laminar flow) can be realized by choosing a suitably high diameter D_{H}. Also a low surface roughness is advantageous to shift the laminar-turbulent flow transition to higher values of Re. The kinematic viscosity of air at 20° C. is about 0.154 cm²/s. The lower limit of D_{H} is therefore given by the relation

$${D}_{H} > 1375 \frac{min}{L} \cdot \frac{Q}{{Re}_{krit}} \cdot mm$$

Each of the gas sources 12, 14, 16 may be formed as gas a cylinder in which the respective gas or gas mixture is provided. For example, the first gas source 12 for nitric oxide comprising gas may comprise pure nitric oxide or a mixture of nitric oxide in nitrogen, whereas the second gas source 14 for oxygen may comprise pure oxygen or air. Additionally, the third gas source 16 for an inert gas may preferably comprise nitrogen.

In an especially preferred embodiment, pure nitric oxide is provided in the first gas source 12, pure oxygen may be provided in the second gas source 14, and nitrogen may be provided in the third gas source 16. This allows adjusting for example the concentration of the oxygen comprising gas and of the nitric oxide comprising gas according to the desired application without further modifications and in any situation even during performing the method. In detail, the oxygen comprising gas may be formed of pure oxygen and nitrogen and the nitric oxide comprising gas may be formed of nitric oxide and nitrogen, whereas the inert gas is nitrogen.

Therefore, flow regulating devices 24, 26, 28 may be provided between each of the gas sources 12, 14, 16 and the conduit 18, or the gas introduction section 20.

According to the invention, well defined pulse sequences of gases are introduced into the conduit 18, or the gas introduction section 20, respectively. Therefore, the arrangement 10 further comprises a controlling device 30 for controlling the regulation devices 24, 26, 28 such, that a stream of gas is generated having the required pulse sequences.

One embodiment of an exemplary defined pulse sequence is shown in FIG. 2. According to FIG. 2, an intermittent and thus discontinuous flow or stream, respectively of oxygen comprising gas, for example pure oxygen, is provided being marked as line a). The stream a) has at least one gap. In this gap, or during this gap, respectively, a defined pulse of nitric oxide comprising gas, for example a mixture of nitrogen and nitric oxide, is provided. This pulse is marked as line c). It can furthermore be seen that the pulse of nitric oxide comprising gas is embedded in a first pulse of inert gas being provided before the pulse of nitric oxide comprising gas and a second pulse of inert gas being provided after the pulse of nitric oxide comprising gas, the first pulse of inert gas and the second pulse of inert gas being provided in the gap of the intermittent stream of oxygen comprising gas. The pulses of inert gas are marked as line b) and may be formed of nitrogen. Advantageously, there is no gap, or time, between the respective pulses or gas phases, respectively. This shall mean that, according to FIG. 2, a pulse of oxygen comprising gas is directly followed by a pulse of inert gas directly after which a pulse of nitric oxide comprising gas is following. Consequently, the pulse of nitric oxide comprising gas is directly followed by a pulse of inert gas, directly after which a pulse of oxygen comprising gas is provided.

It has to be noted that for a plurality of applications, a rather long pulse, or stream, of oxygen comprising gas is provided which is shortly interrupted for an insertion of the inert gas and the nitric oxide comprising gas. However, the exact duration of the interruption, or the gap, respectively, and thus the duration and length of the respective pulses is adjusted in dependence of the desired application.

The method according to the invention using, for example, pulse sequences like shown in FIG. 2 reduces or completely avoids the danger of oxygen mixing or coming in contact with nitric oxide and thus reduces or completely avoids the formation of nitrogen oxides in higher oxidation states, for example nitrogen dioxide.

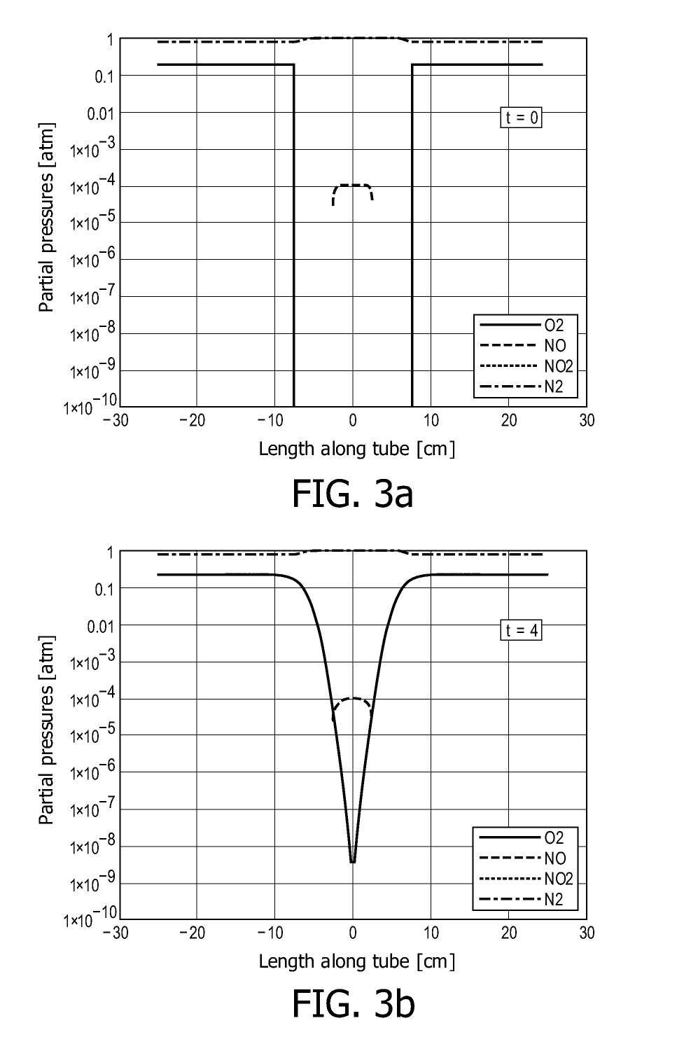

The effect of the method according to the invention can be visualized in the diagrams according to FIG. 3. In detail, the diagrams of FIG. 3 are based on an intermittent flow of air, in which gap two pulses of nitrogen are provided, in which a pulse of a mixture of nitric oxide and nitrogen is provided. The diagrams show the partial pressures of respective gases along a tube in which the gas stream is conveyed. The total pressure is 1 atm. The gas flow is assumed to be non-turbulent, i.e. the Reynolds number is assumed to be ≦2300 which may be translated into a corresponding relation between the diameter of the duct D_{H} and the volumetric flow rate Q D_{H}≧0.598 mm·Q[L_{SATP}/min]; the gas flow velocity V is then

$$V = \frac{Q}{π / 4 \cdot {D}_{H}^{2}}$$

Under these conditions of a laminar flow and homogeneity across the cross-section of the air duct (i.e. constant flow velocity and partial pressures) the (axial) partial pressure profiles are determined by axial diffusion and the nitrogen dioxide formation reaction 2NO+O₂→2NO₂. The axial co-ordinates given in the following paragraphs are measured within a co-ordinate system which is moving with the gas flow, i.e. with axial velocity V.

FIG. 3a shows the conditions at t=0, i.e. directly after forming the pulse sequence. Until a length of −7.5 cm, both oxygen and nitrogen are present which represents the presence of air, i.e. the oxygen comprising gas pulse. At −7.5 cm, the partial pressure of oxygen falls to zero, whereas the partial pressure of nitrogen rises to 1 atm. This shows the presence of pure nitrogen, i.e. the first inert gas pulse. From −2.5 cm to +2.5 cm, the pulse of nitric oxide can be seen. The partial pressure of nitrogen still lies in the range of 1 atm, because the concentration of nitric oxide lies in the range of only 1×10⁻⁴ atm, the rest being nitrogen. From about +2.5 cm to +7.5 cm, only nitrogen is present with a partial pressure of approximately l atm which represents the second inert gas pulse. At +7.5 cm, the concentration of oxygen rises and the concentration of nitrogen falls to the respective concentrations of air indicating the oxygen comprising gas.

FIG. 3b shows the conditions at t=4, i.e. after 4 seconds of streaming, for example, through the conduit 18. It can be seen that the respective gases, in particular oxygen, diffuse through the phase borders into the adjacent pulse. In particular in the range of about 0 cm, it can be seen that oxygen diffuses into the nitric oxide pulse only in very limited concentrations, e.g. having a partial pressure in the range of 10⁻9 atm. These concentrations however are far too low to allow a reaction of nitric oxide and oxygen to form nitrogen oxides at a time, before a patient absorbs the gas stream or exhausts it. This can be seen by the fact that no nitrogen dioxide, for example is formed.

The diagrams of FIG. 3 indicate the behavior of the generated gas stream being transported to a patient and furthermore into the patient and through the trachea to the lungs. The diagram at t=0 again shows the behavior directly after the generation of the gas stream comprising the respective pulse sequence. At a time tₐ, for example, the gas stream is flowing through the tube and is reaching the patient at a time t_{b}. The gas stream will be further conveyed to the trachea at a time t_{c} and will finally reach the lungs, or the alveoli, respectively, at a time t_{d} and will be absorbed by the human body. With respect to FIG. 3b, the conditions of t=4 may be taken into consideration when thinking about t_{d}, as a breathing cycle mostly is finished after 4 seconds. From the above it can be seen that at a time from generating the gas stream until the respective gases are absorbed by the human body, the danger of a reaction of nitric oxide with oxygen to nitrogen oxides in higher oxidation states is strongly reduced or completely avoided.

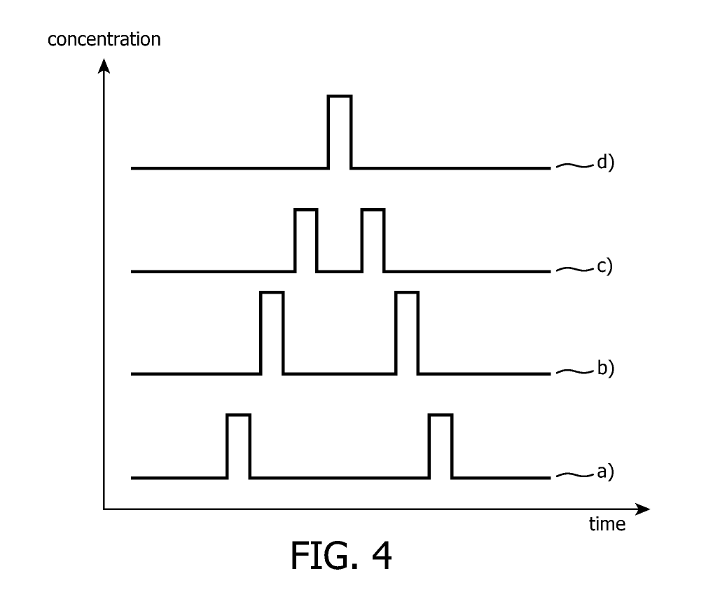

In FIG. 4, a further pulse sequence for a method according to the invention is shown. According to FIG. 4, the oxygen comprising gas is air and is marked as line a). During the gaps of the stream of air, again, a pulse of nitric oxide comprising gas is introduced (line d) which is embedded in two pulses of inert gas, for example nitrogen. The inert gas pulses are marked as line c). Additionally, further pulses of gas are inserted during the gap of the oxygen comprising gas according to line a). These additional pulses may comprise pure oxygen and are located directly before the first inert gas pulse, and directly after the second inert gas pulse, respectively and are visualized by line b). Again, it is preferred to provide each pulses directly one after the other in order to generate a continuous stream of gas.

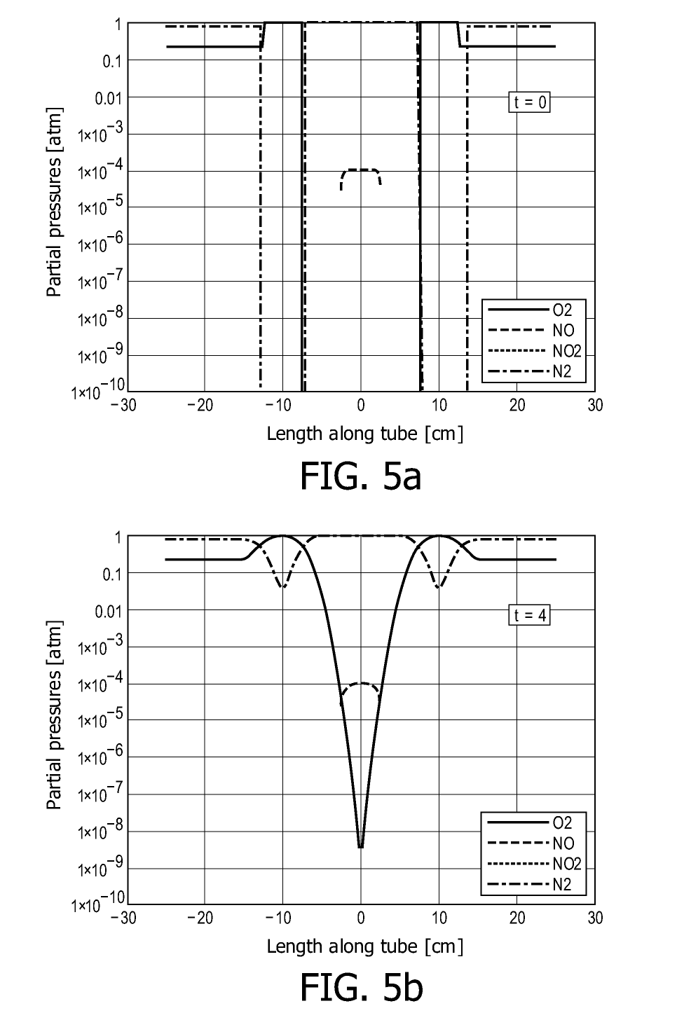

In FIG. 5, the effect of a pulse sequence corresponding to FIG. 4 is shown. The circumstances of FIG. 5 are comparable to the ones being used in FIG. 3. However, the pulse sequence of FIG. 5 comprises a pulse of air, followed by a pulse of oxygen, after which two inert gas pulses of nitrogen embedding a single pulse of nitric oxide are provided. The second pulse of nitrogen is then followed by a further pulse of oxygen after which again, a pulse of air follows. Again, in FIG. 5a the conditions at t=0 are shown. Up to −12.5 cm, the respective concentrations indicate the stream of air, i.e. the oxygen comprising gas. At −12.5 cm, the concentration of nitrogen falls to zero, as a pulse of pure oxygen follows. At −7.5 cm, the partial pressure of nitrogen rises to about 1 atm and the partial pressure of oxygen falls to zero, as a pulse of pure nitrogen is following, i.e. the first inert gas pulse. From −2.5 cm to +2.5 cm, the pulse of nitric oxide can be seen. The concentration of nitrogen still lies in the range of 1 atm, because the concentration of nitric oxide lies in the range of only 1×10⁻⁴ atm only, the rest being nitrogen. At +7.5 cm, the concentration of nitrogen again drops to zero and the concentration of oxygen rises to result in a partial pressure of 1 atm indicating a pulse of pure oxygen. Subsequently, the partial pressure of oxygen falls and the partial pressure of nitrogen rises to result in the respective concentrations of air at approximately +12.5 cm, indicating the oxygen comprising gas.

FIG. 5b again shows the conditions at t=4, i.e. after 4 seconds of streaming through the conduit. Again, it can be seen that especially oxygen may diffuse through the pulse borders. However, the concentration is too low to allow a generation of nitrogen dioxide, for example, in a time being critical for most applications, even for therapeutic applications. For example, at a point at 0 cm, the partial pressure of oxygen lies in a range of 10⁻⁹ atm, whereas the concentration of nitric oxide stays nearly unchanged.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. Method for generating a stream of gas, the gas comprising oxygen and nitric oxide, wherein
an intermittent stream of oxygen comprising gas having at least one gap is provided, in which gap
a pulse of nitric oxide comprising gas is provided, wherein
the pulse of nitric oxide comprising gas is embedded in a first pulse of inert gas being provided before the pulse of nitric oxide comprising gas and a second pulse of inert gas being provided after the pulse of nitric oxide comprising gas, the first pulse of inert gas and the second pulse of inert gas being provided in the gap of the intermittent stream of oxygen comprising gas.

2. Method according to claim 1, wherein the stream of gas is provided in a gas administration device (22) for therapeutic applications.

3. Method according to claim 1, wherein the gas stream exhibits a Reynolds number of ≦4000.

4. Method according to claim 1, wherein air or oxygen is used as oxygen comprising gas.

5. Method according to claim 4, wherein the oxygen comprising gas is air and wherein a first pulse of oxygen is provided before the first inert gas pulse and wherein a second pulse of oxygen is provided after the second inert gas pulse, the first and the second oxygen pulses being provided in the gap of air.

6. Method according to claim 1, wherein nitrogen is used for providing the first and the second inert gas pulse.

7. Method according to claim 1, wherein a mixture of nitrogen and nitric oxide is used as nitric oxide comprising gas.

8. Method according to claim 1, wherein the gas stream exhibits a SATP flow rate in the range of ≧0,01 L_{SATP}/min to ≦10 L_{SATP}/min.

9. Method according to claim 1, wherein the pulse width Wₚᵤₗₛₑ of each inert gas pulse lies in the range of ≧1 cm to ≦10 cm.

10. Arrangement for providing an oxygen and nitric oxide comprising gas stream, the arrangement (10) comprising
a first gas source (12) for a nitric oxide comprising gas,
a second gas source (14) for an oxygen comprising gas, and a
a third gas source (16) for an inert gas, wherein
each of the first gas source (12), the second gas source (14), and the third gas source (16) are connected to a conduit (18), wherein
a flow regulation device (24, 26, 28) is provided for each gas source (12, 14, 16) to allow a flow of the gas into the conduit (18) to be adjusted, and wherein
the arrangement (10) further comprises a controlling device (30) for controlling the flow regulation device (24, 26, 28) such, that a stream of gas is generated according to a method according to claim 1.
